(19) United States
(12) Patent Application Publication
(10) Pub. No.: US 2014/0296871 A1
(43) Pub. Date: Oct. 2, 2014

(54) SURGICAL GUIDING AND POSITION SYSTEM

(71) Applicants: Chieh-Hsiao Chen, Taichung City (TW); Shinn-Zong Lin, Taichung City (TW); Horng-Jyh Harn, New Taipei City (TW)

(72) Inventors: Chieh-Hsiao Chen, Taichung City (TW); Shinn-Zong Lin, Taichung City (TW); Horng-Jyh Harn, New Taipei City (TW)

(21) Appl. No.: 14/229,620

(22) Filed: Mar. 28, 2014

Related U.S. Application Data
(60) Provisional application No. 61/807,041, filed on Apr. 1, 2013.

(51) Int. Cl. A61B 19/00

(52) U.S. Cl. CPC A61B 19/2203
USPC 606/130

(57) ABSTRACT

The present invention discloses a surgical guiding and positioning system, comprising: a global information device, a local information device, and a computing device. The global information device is capable of collecting an overall information of a surgical site before a surgical procedure begins. The local information device can comprise a surgical device including at least one sensor and provide a real-time information of a section of the surgical site. The computing device is capable of calculating a position data of the surgical device in the surgical site by comparing the overall information and the real-time information. Therefore, the present invention is capable of providing the real-time guidance and monitoring during a surgical procedure so that the surgeon can acquire precise real-time information of the surgical site and perform the surgery efficiently.

### BACKGROUND OF THE INVENTION

1. Field of the Invention

The present invention relates to a guiding and positioning system, and more particularly, to a surgical guiding and positioning system for real-time guidance and monitoring.

2. Background

Common brain diseases, such as brain tumors, Parkinson's disease (PD) and epilepsy, often cause the following symptoms: tremor, headache, vomiting, vision impairment, impaired state of consciousness or paralysis, etc. Therefore, these diseases not only adversely affect the patients' quality of life but sometimes can directly contribute to the patients' death. Invasive surgical procedures are usually performed after conservative treatments, such as medicines or physical therapies, failed to relieve the patients' symptoms. A surgeon has to select an extremely small area of the cranial nerves to perform the thermal ablation procedure with a surgical probe.

Currently, a surgeon can only rely on the pre-operative data before performing a brain surgery, but even the minor shift of the brain during the surgical procedure or the improper operation of the stereotactic positioning system often renders the position data of the surgical site inaccurate.

The existing surgical navigation systems include, for example, MRI (Magnetic resonance imaging) guided navigation systems, CT (Computer Tomography) guided navigation systems, X-ray-guided navigation systems and neural discharge recording apparatuses. Installing the MRI or CT-guided navigation system in the operation room is not cost-effective, and operating the system in the limited surgical space is inconvenient as well. The X-ray fluoroscopy data are not reliable, and it is not easy to clearly record the data with the neural discharge recording apparatus. Moreover, the position where the chip is embedded is usually undesirable.

Even if a surgeon employs the frame technique to provide the landmark or perform the navigation function (see FIG. 1 in which a frame 1 is shown), it is possible that the pre-operative planned pathway may change due to a number of factors, such as the patient's position, the frame fixation, or the surgeon's skills, and result in an increase in the complication or mortality rate.

Therefore, there is a need in the art for a surgical guiding and positioning system capable of providing the real-time guidance and monitoring during the surgical procedure so that the surgeon can acquire precise real-time information of the surgical site and perform the surgical procedure efficiently.

### SUMMARY OF THE INVENTION

One object of the present invention is to provide a surgical guiding and positioning system capable of providing the real-time guidance and monitoring during the surgical procedure so that the surgeon can acquire precise real-time information of the surgical site and perform the surgical operation efficiently.

According to the object of the present invention, there is provided a surgical guiding and positioning system, comprising: a global information device, a local information device and a computing device. The global information device is capable of collecting overall information of a surgical site before a surgical procedure begins. The local information device can comprise a surgical device including at least one sensor, wherein the at least one sensor is configured to collect a set of data within the surgical site during the surgical procedure. The computing device is capable of calculating a position data of the surgical device in the surgical site based on the overall information and the collected set of data.

According to the object of the present invention, there is further provided a surgical guiding and positioning system, comprising: a global information device, a local information device, a computing device, a display device, a warning device and an operating device. The global information device is capable of collecting overall information of a brain before a surgical procedure begins. The local information device can comprise a surgical device including at least one sensor, wherein the at least one sensor is configured to collect a set of data within the brain. The computing device is capable of calculating a position data of the surgical device in the brain based on the overall information and the collected set of data. The display device is capable of displaying a real-time position of the surgical device in a 3D image derived from the overall information. The warning device is capable of sending out a signal in response to the surgical device having detected, for example, a blood vessel or vital area in front of it. The operating device is capable of controlling the operation of the surgical device.

According to the present invention, the at least one sensor is a position sensor selected from at least one of the following: an optical sensor, an acoustic wave sensor, a mechanical wave sensor, a G-Sensor, a magnetic sensor, a microwave sensor, a laser sensor, an electrode sensor and any combination thereof.

According to the present invention, the position sensor is an ultrasound sensor configured to collect a relative reference position, a distance information or an image information.

According to the present invention, the position sensor is an OCT (Optical Coherence Tomography) sensor.

According to the present invention, the at least one sensor provides the same kind or different kinds of signals, such as OCT signals, electrical impedance signals, echo signals, and any combination thereof.

According to the present invention, the at least one sensor can be disposed in any position at the surgical device, such as the front end, the rear end or the combination thereof.

According to the present invention, the at least one sensor is disposed in the same or different positions at the surgical device.

According to the present invention, the local information device is configured to sense the positions of tissues surrounding a blood vessel or the position of the at least one sensor.

According to the present invention, the surgical device is received in an outer sheath on which the at least one sensor is disposed.

In an embodiment of the present invention, the surgical guiding and positioning system further comprises a monitoring device configured to issue a real-time signal in response to the surgical device being in a target position or a risky position or deviates from the planned surgical pathway. According to the present invention, the local information device is controlled by a surgeon or a robotic arm operated by the surgeon, and the movement of the local information device is monitored by the monitoring device, which is capable of sending out a warning to the surgeon during operation under certain circumstances

According to the present invention, the one-step displacement of the robotic arm is at a millimeter scale or an even smaller scale.

According to the present invention, the global information device, the local information device, the computing device and the monitoring device operate automatically through a predetermined planning.

In an embodiment of the present invention, the surgical guiding and positioning system further comprises a display device configured to display a real-time position of the surgical device in a 3D image based on the position data, wherein the 3D image is derived from the overall information.

The aforementioned aspects and other aspects of the present invention will be better understood by reference to the following exemplary embodiments and drawings.

### BRIEF DESCRIPTION OF THE DRAWINGS

FIG. 1 is a schematic view showing a prior art frame in the state of being used.

FIG. 2 is a block diagram showing the configuration of a surgical guiding and positioning system in accordance with an embodiment of the present invention.

FIG. 3 is a schematic view showing the arrangement of a sensor in accordance with an embodiment of the present invention.

FIG. 4 is a schematic view showing the arrangement of one or more sensors in accordance with another embodiment of the present invention.

FIG. 5 is a schematic view showing the arrangement of a plurality of sensors in accordance with another embodiment of the present invention.

FIG. 6 is a schematic view showing the arrangement of a plurality of sensors in accordance with another embodiment of the present invention.

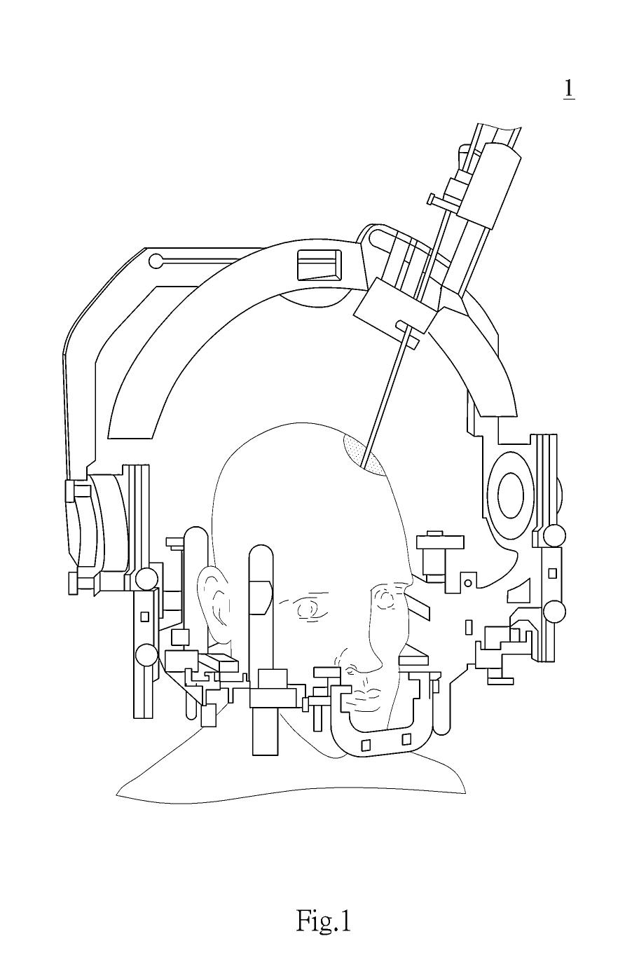

### DETAILED DESCRIPTION OF THE PREFERRED EMBODIMENTS

While this invention will be fully described with preferred embodiments by reference to the accompanying drawings, it is to be understood beforehand that those skilled in the art can make modifications to the invention described herein and attain the same effect, and that the description below is a general representation to those skilled in the art and is not intended to limit the scope of the present invention.

Throughout the disclosure, the term “surgical device” and “surgical instrument” are used interchangeably, and they broadly refer to tools or devices that can be used in a surgery or operation to achieve desired effects.

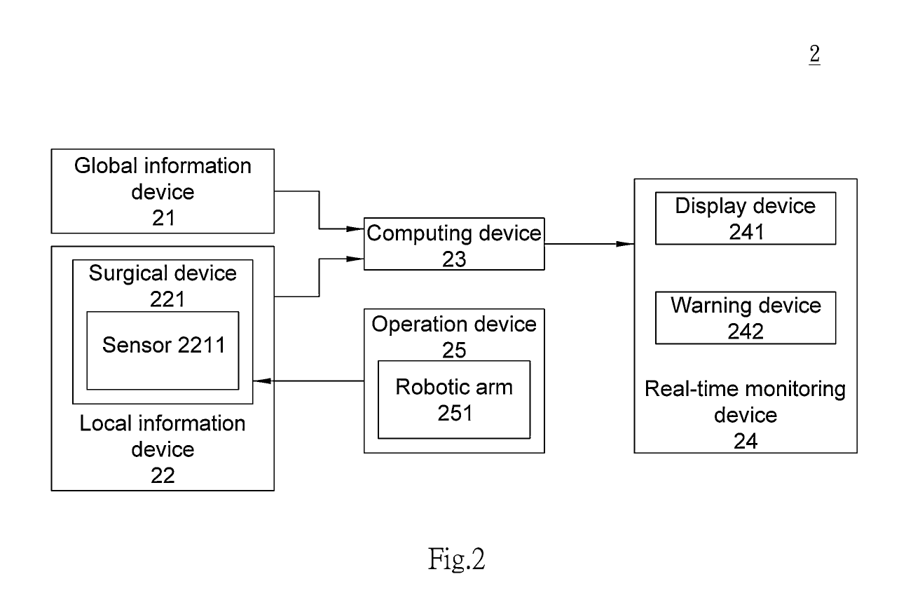

FIG. 2 is block diagram showing the configuration of a surgical guiding and positioning system in accordance with an embodiment of the present invention. The configuration and operation of the surgical guiding and positioning system of the present invention will be briefly described by reference to FIG. 2. As shown in FIG. 2, the embodiment of the surgical guiding and positioning system 2 of the present invention mainly comprises a global information device 21, a local information device 22, a computing device 23, a real-time monitoring device 24 and an operating device 25, which are coupled and communicate with each other.

The global information device 21 is capable of collecting overall information of a surgical site before a surgical procedure begins. In a preferred embodiment, the surgical site can be, but not limited to, a brain. In a preferred embodiment, the overall information can be acquired through CT, MRI, surface scan, X-ray scan, ultrasound scan, and etc. However, in real practice, the way of acquiring the overall information is not limited to the aforementioned examples. Therefore, a surgeon can learn the overall information (e.g. the intracranial anatomy, the target or lesion location, or the surface land markings) of the surgical site before a surgical procedure begins and plan a surgical pathway beforehand.

According to an embodiment of the present invention, the local information device 22 comprises a surgical device 221, which includes at least one sensor 2211 that can be directly disposed on the surgical device 221 or an outer sheath (not shown in FIG. 2) of the surgical device 221. The arrangement of the surgical device 221 and at least one sensor 2211 will be detailed below.

In a preferred embodiment, examples of the surgical device 221 include a biopsy needle, a biopsy forceps, a clamp, a laser fiber, a brain pressure monitor catheter, and etc. However, in real practice, the surgical device 221 is not limited to the aforementioned examples. Moreover, a surgeon can control the surgical device 221 via the operating device 25. In a preferred embodiment, it is preferable that the operating device 25 comprises a robotic arm 251 through which the surgeon can control the operation of the surgical device 221. The one-step displacement of the robotic arm 251 is at a precise and stable millimeter scale (e.g. 0.1 cm) or an even smaller scale.

During the surgical procedure, the local information device 22 can regionally sense the surgical site and provide relevant real-time information. The at least one sensor 2211 can be directly disposed on the surgical device 221 according to practical needs. However, the least one sensor 2211 is not limited to the aforementioned arrangement in other embodiments.

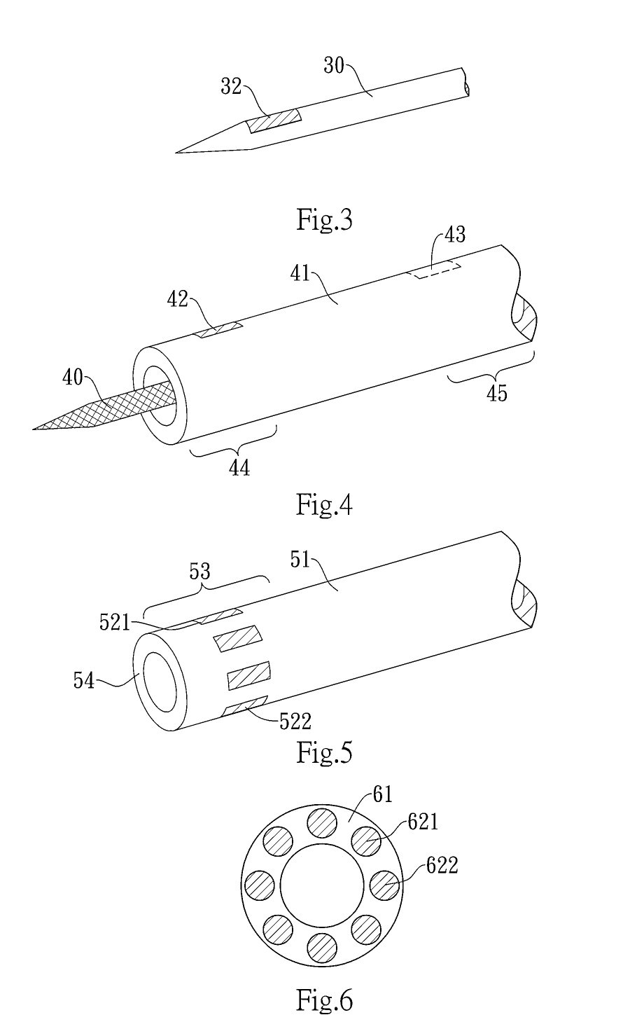

FIG. 3 is a perspective view showing the arrangement of a sensor in accordance with an embodiment of the present invention. According to the present invention, the local information device mainly includes a surgical device including at least one sensor, which constructs the surgical apparatus of the present invention. In an embodiment of the surgical apparatus as shown in FIG. 3, at least one sensor 32 is directly disposed on a surgical device 30 to instantly and regionally sense the surgical site with the movement of the surgical device 30, thereby providing relevant information. As described above, examples of the surgical device 30 include, but not limited to, a biopsy needle, a biopsy forceps, a clamp, a laser fiber, a brain pressure monitor catheter, and etc. The at least one sensor 32 is a position sensor selected from at least one of the following: an optical sensor, an acoustic wave sensor, a mechanical wave sensor, a G-Sensor, a magnetic sensor, a microwave sensor, a laser sensor, an electrode sensor and any combination thereof. Additionally, the number and type of the at least one sensor 32 are not limited to those shown in the drawings. In a preferred embodiment, a plurality of sensors 32 can be provided on the surgical device 30 to simultaneously sense the surgical site and provide a variety of relevant information.

In the present invention, at least one sensor can be disposed on an outer sheath of the surgical device, in addition to the arrangement of the sensor directly on the surgical device. FIG. 4 is a schematic view showing the arrangement of one or more sensors in accordance with another embodiment of the present invention. As shown in FIG. 4, a surgical device 40 is received in or detachably coupled to a chamber of an outer sheath 41. Moreover, the surgical device 40 is wrapped by the outer sheath 41 and is movable therein. In this embodiment, a first sensor 42 is disposed on the outer circumference surface of a first end portion 44) of the outer sheath 41, and a second sensor 43 is disposed on the outer circumference surface of a second end portion 45. The two sensors 42 and 43 are configured to sense the surgical site and provide relevant real-time information. In order to construct a 3D image based on the real-time information provided by a single sensor 42, the sensor 42, through the rotation of the outer sheath 41, can continuously sense signals from at least two different areas at least two different time points, thereby constructing real-time local information of the surgical site. In other words, the outer sheath 41 is rotatable or movable with respect to the surgical device 40 so as to cause rotation or movement of the sensors 42 and 43 and make a change in the relative position. For example, the rotation or movement of the outer sheath 41 with respect to the surgical device 40 can be realized through hydraulic or pneumatic means. In another aspect, the outer sheath 41 can have a plurality of chambers to receive different surgical devices 40. By means of the present invention, the surgical apparatus configured in such a manner is advantageous in that different data within the surgical site can be collected during an operation involving the surgical site, and such data can be processed to generate information that may be useful for the operation.

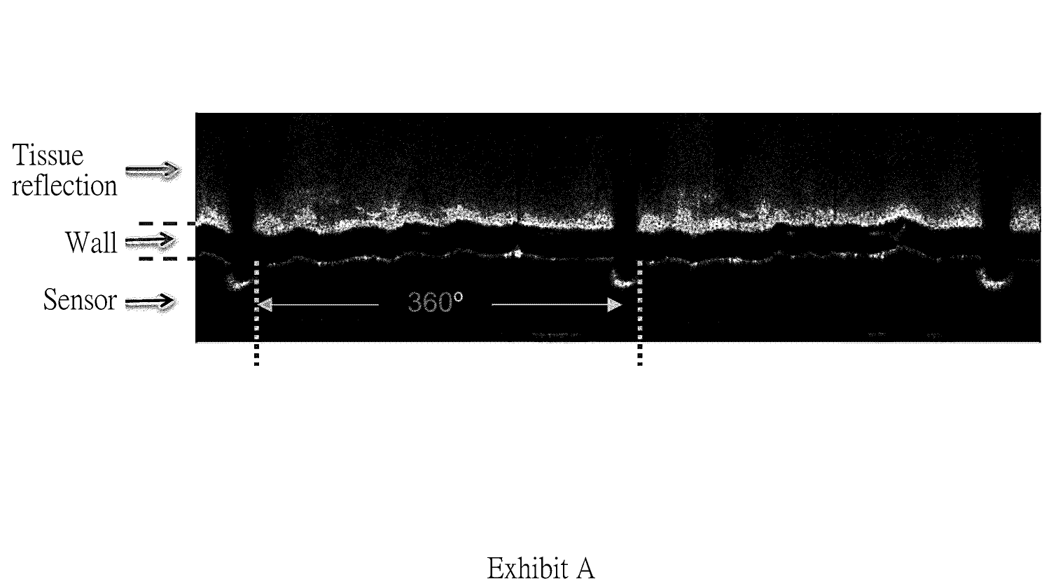

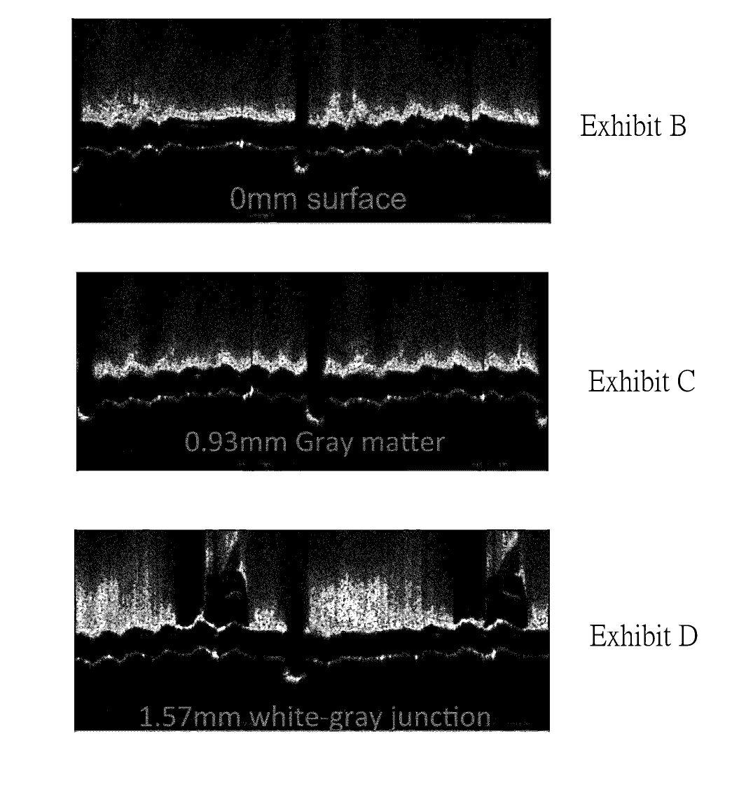

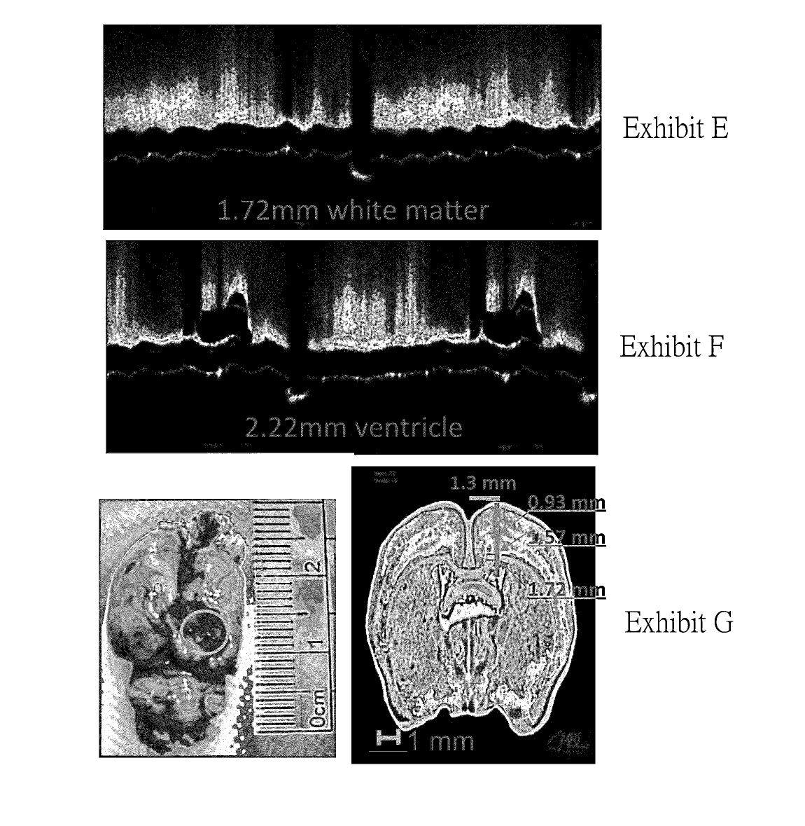

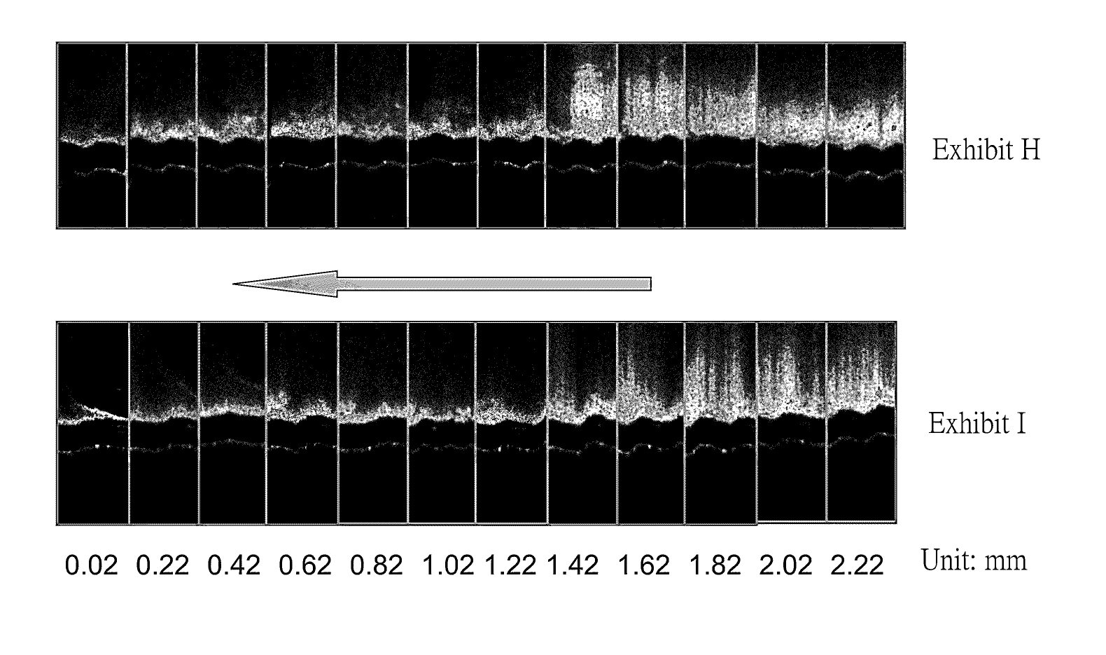

FIG. 5 is a schematic view showing the arrangement of a plurality of sensors 521 and 522 in accordance with another embodiment of the present invention. The embodiment illustrated in FIG. 5 is different from the one shown in FIG. 4 in that the plurality of sensors 521 and 522 are disposed on the outer circumference surface of an end portion 53 of the outer sheath 51 having ring-like sectional surface 54 to simultaneously sense multiple signals in a stop position, thereby constructing a 3D image of the surgical site. In the present invention, the positions where the pluralities of sensors are arranged are not limited to those on the exterior of the outer sheath. As shown in FIG. 6, the plurality of sensors 621 and 622 can be disposed in the same or different positions on the ring-like sectional surface 61.

In the embodiments shown in FIGS. 4-6, the sensor(s) is disposed on the outer sheath of the surgical device. Such an arrangement is advantageous in that a variety of surgical devices can be placed inside the outer sheath according to needs without rearranging the sensor(s), thereby the real-time information related to the surgical site can be provided continuously. For general surgical applications, the surgical site is not limited to any part or organ of the human body. In a preferred embodiment, the surgical site can be a brain, and the relevant real-time information can be the same kind or different kinds of signals, such as OCT signals, electrical impedance signals, echo signals, position data of tissues surrounding a blood vessel or the plurality of sensors, and etc.

Furthermore, the plurality of sensors disposed on the surgical device or the outer sheath can be position sensors employing optical imaging techniques or frequency range sensing techniques, such as, for example, but not limited to, optical sensors, acoustic wave sensors, mechanical wave sensors, G-Sensors, magnetic sensors, microwave sensors, laser sensors, electrode sensors and any combination thereof. In an embodiment of the present invention, said position sensor is an OCT sensor or an ultrasound sensor.

In real practice, the positions where the plurality of sensors are arranged are not limited to those at the surgical device or the outer sheath's front end, rear end, or any combination thereof. The plurality of sensors can be disposed at any positions where relevant real-time information of the surgical site can be provided during the surgical procedure.

In the present invention, the type of sensor is selected based on the position where the sensor is to be disposed and the resolution required. For example, it is preferable that an OCT sensor is disposed at the front end of the surgical device or the outer sheath to instantly provide high resolution images of the surgical sites (at a scale even smaller than a millimeter scale) during the surgical procedure. Moreover, the ultrasound sensor can instantly provide high quality and wider range ultrasound signals (inclusive of image and distance information) of the surgical site, and is configured to sense a relative reference position of the front end of the surgical device or the outer sheath within the surgical site. Similarly, the position and type of the sensor are not limited to the aforementioned examples. For example, optical waves, sound waves, laser or any technique utilizing the Doppler Effect can be used to sense a relative reference position of the front end of the surgical device or the outer sheath within the surgical site.

Please refer back to FIG. 2. The computing device 23 is capable of calculating a position data of the surgical device in the surgical site by comparing the overall information collected by the global information device 21 before the surgical procedure begins and the relevant real-time information sensed and provided by the local information device 22 during the surgical procedure. In an embodiment, the computing device 23 is capable of calculating a position data of the surgical device in the surgical site, such as a brain (within the cranium), by comparing an overall information, such as CT imaging data, MRI imaging data, surface scan data or X-ray data, collected by the global information device 21 before the surgical procedure begins and relevant real-time information, i.e. the same kind or different kinds of signals, such as OCT signals, electrical impedance signals, echo signals, and position data of tissues surrounding a blood vessel or the plurality of sensors, sensed and provided by the local information device 22 during the surgical procedure.

Moreover, the computing device 23 further calculates the properties of the position data of the surgical device in the surgical site. In real practice, the computing device 23 is capable of combining the aforementioned high resolution images (at a millimeter scale or an even smaller scale) or high quality and wider range ultrasound signals of the surgical site based on the overall information, such as the CT imaging data, MRI imaging data, surface scan data or X-ray data, collected by the global information device 21 before the surgical procedure begins. Alternatively, the computing device 23 is capable of locating the exact position of the front end of the surgical device 221 or the outer sheath (not shown in FIG. 2) within the surgical site by combining a relative reference position of the front end of the surgical device 221 or the outer sheath (not shown in FIG. 2) within the surgical site sensed by the sensor 2211 employing the ultrasound technique based on the original position of the surgical site. Therefore, the real-time guidance and monitoring can be provided during a surgical procedure and this allows the surgeon to acquire precise real-time information of the surgical site and perform the surgical procedure efficiently, thereby reducing the probability of causing injuries to the patient due to incorrect position data.

Please refer back to FIG. 2. In a preferred embodiment, the real-time monitoring device 24 can comprise a display device 241 and a warning device 242. The display device 241 is capable of displaying a 3D image in its display screen based on an overall information, such as CT imaging data, MRI imaging data, surface scan data or X-ray data, collected by the global information device 21 before the surgical procedure begins, wherein the 3D image can be derived from the overall information. During the surgical procedure, the display device 241 is capable of displaying a real-time position of the surgical device 221 in the 3D image based on the position data calculated by the computing device 23. Therefore, the surgeon can learn the position of the surgical device 221 through the real-time monitoring and adjust the position of the surgical device 221 according to needs.

In a preferred embodiment, the warning device 242 is capable of sending out a real-time warning to the surgeon when the surgical device 221 detects a blood vessel or vital area in front of it, or when the surgical device 221 is in a target position or a risky position or deviates from the planned surgical pathway. In another preferred embodiment, the real-time monitoring device 24 can be controlled by the surgeon or the robotic arm 251 operated by the surgeon according to needs.

In addition, the global information device 21, the local information device 22, the computing device 23, the real-time monitoring device 24 and the operating device 25 included in the embodiment of the surgical guiding and positioning system 2 of the present invention can operate automatically through a predetermined planning under the supervision of the surgeon. In other words, the surgeon can make a detailed surgical planning before performing a surgical procedure and set the surgical guiding and positioning system 2 of the present invention to perform the surgical procedure automatically. During the surgical procedure, the surgeon can monitor the progress of the surgical procedure, provide guidance at a real-time basis, and make suitable changes during the course of the surgical procedure. Such conception of the present invention can be realized by an automatic robotic arm having a one-step displacement at a precise and stable millimeter scale (e.g. 0.1 cm) or an even smaller scale.

Those having ordinary knowledge in the art understand that the examples of overall information collected by the global information device 21 before the surgical procedure begins, the examples of relevant real-time information provided by the local information device 22 and the embodiments of the sensor 2211 and the real-time monitoring device 24 are exemplary and are not intended to limit the present invention. Any system without departing from the spirit and scope of the surgical guiding and positioning system of the present invention shall fall within the scope of the present invention.

In a preferred embodiment, the operating device 25 can be automated. That is, the global information device 21, the local information device 22, the computing device and the real-time monitoring device in the surgical guiding and positioning system of the present invention operate automatically through a predetermined planning.

Exhibits A˜I show the results obtained by actually operating a surgical guiding and positioning system of the present invention. As can be seen from Exhibits A˜I, the OCT images can provide information of the depth in the white matter, gray matter or ventricle. The results prove that the relevant real-time information sensed and provided by the local information device 22 during the surgical procedure can be used to determine the real-time intracranial position.

In sum, the surgical guiding and positioning system of the present invention can provide the surgeon with the real-time guidance and monitoring during the surgical procedure so that the surgeon can acquire precise real-time information of the surgical site and perform the surgical operation efficiently. In addition, the surgical guiding and positioning system of the present invention is applicable to, for example, the liver surgery, brain endoscopic surgery and all endoscopic biopsy and endoscopic surgeries in the operation room, prostate and urinary bladder endoscopic surgeries, otolaryngological endoscopic surgery, gastroscopic surgery, colonoscopic surgery, thoracoscopic surgery, laparoscopic surgery, endovascular surgery, dental implants, root canal treatment, etc.

The surgical guiding and positioning system of the present invention has been described above by way of preferred embodiments by reference to the accompanying drawings. All the features disclosed in this specification may be combined with other methods. Each feature disclosed in this specification may be replaced by an alternative feature serving the same, equivalent, or similar purpose. Thus, except for those particularly distinctive features, each feature disclosed herein is only an example of a generic series of equivalent or similar features. Given the above description of preferred embodiments, those skilled in the art would understand that the present invention features several aspects of novelty and inventive step over the prior art and is industrially applicable. Various modifications and substitutions may be made by those skilled in the art without departing from the spirit and scope of the present invention.

What is claimed is:

1. A surgical guiding and positioning system, comprising:
a global information device configured to collect an overall information of a surgical site before a surgical procedure begins;
a local information device including a surgical device which includes at least one sensor, wherein the at least one sensor is configured to collect a set of data within the surgical site during the surgical procedure; and
a computing device configured to calculate a position data of the surgical device in the surgical site based on the overall information and the collected set of data.

2. The surgical guiding and positioning system according to claim 1, wherein the at least one sensor is a position sensor selected from at least one of the following: an optical sensor, an acoustic wave sensor, a mechanical wave sensor, a G-Sensor, a magnetic sensor, a microwave sensor, a laser sensor, an electrode sensor and any combination thereof.

3. The surgical guiding and positioning system according to claim 2, wherein the position sensor is an ultrasound sensor configured to collect a relative reference position, a distance information, or an image information.

4. The surgical guiding and positioning system according to claim 2, wherein the position sensor is an OCT (Optical Coherence Tomography) sensor.

5. The surgical guiding and positioning system according to claim 2, wherein the at least one sensor provides the same kind or different kinds of signals selected from the group consisting of OCT signals, electrical impedance signals, echo signals, and any combination thereof.

6. The surgical guiding and positioning system according to claim 1, wherein the at least one sensor is disposed at a front end, a rear end, or any combination thereof of the surgical device.

7. The surgical guiding and positioning system according to claim 1, wherein the at least one sensor is disposed at the same or different positions on the surgical device.

8. The surgical guiding and positioning system according to claim 1, wherein the local information device is configured to sense the positions of tissues surrounding a blood vessel or the at least one sensor.

9. The surgical guiding and positioning system according to claim 1 further comprising:
a monitoring device sending out a signal in response to the local information device being in a target position or a risky position or deviates from a planned surgical pathway.

10. The surgical guiding and positioning system according to claim 9, wherein the local information device is controlled by a surgeon or a robotic arm operated by the surgeon.

11. The surgical guiding and positioning system according to claim 10, wherein the one-step displacement of the robotic arm is at a millimeter scale or an even smaller scale.

12. The surgical guiding and positioning system according to claim 10, wherein the global information device, the local information device, the computing device and the monitoring device operate automatically through a predetermined planning.

13. The surgical guiding and positioning system according to claim 1, wherein the surgical device is received in an outer sheath on which the at least one sensor is disposed.

14. The surgical guiding and positioning system according to claim 1 further comprising a display device displaying a real-time position of the surgical device in a 3D image based on the position data, wherein the 3D image is derived from the overall information.

15. The surgical guiding and positioning system according to claim 1, wherein the surgical site is a brain.

16. A surgical apparatus configured to collect information from a surgical site, comprising:
a surgical instrument for an operation involving the surgical site;
an outer sheath, detachably coupled to the surgical instrument; and
a first sensor and a second sensor disposed on the outer sheath, the surgical instrument, or the combination thereof, wherein the first sensor and the second sensor are configured to collect a set of data while the surgical instrument is in physical contact or in proximity of the surgical site during the operation.

17. The surgical apparatus of claim 16, wherein the first sensor is disposed on a first end portion of the outer sheath, the second sensor is disposed on a second end portion of the outer sheath, and the first end and the second end are two opposite ends of the outer sheath.

18. The surgical apparatus of claim 16, wherein the first sensor and the second sensor are disposed on a same end portion of the outer sheath.

19. The surgical apparatus of claim 18, wherein the first sensor and the second sensor are disposed on an outer circumference surface of an end section of the sheath.

20. The surgical apparatus of claim 16, wherein the outer sheath comprises a ring-like sectional surface on one end of the outer sheath, and the first sensor and the sensor are disposed on the ring-like sectional surface.
